(19) United States
(12) Patent Application Publication
(10) Pub. No.: US 2010/0063437 A1
(43) Pub. Date: Mar. 11, 2010

(54) Multifunctional surgical instrument

(76) Inventors: Drew V. Nelson, San Carlos, CA (US); Thomas H. Hsu, Foster City, CA (US)

Correspondence Address: BELL & ASSOCIATES, 58 West Portal Avenue No. 121, SAN FRANCISCO, CA, 94127, US

(21) Appl. No.: 12/309,309

(22) PCT Filed: Jul. 12, 2007
(86) PCT No.: PCT/US07/15906

§ 371 Date: Jun. 16, 2009

Related U.S. Application Data
(60) Provisional application No. 60/830,238, filed on Jul. 12, 2006.

(51) Int. Cl. A61B 17/00; A61M 1/00

(52) U.S. Cl. 604/35; 606/13; 606/1

(57) ABSTRACT

The invention provides a multi-tool instrument for use in surgical procedures. The tools can be rapidly deployed and retracted during a surgical procedure without requiring exchange of instruments, without compromising the integrity of the anatomical entry site, without obstructing the surgical field of view, and without harming the patient.

### FIELD OF THE INVENTION

The invention relates to instruments that can perform several surgical functions without the need for removing the surgical instrument from the body of the patient.

### BACKGROUND

The use of minimally invasive procedures in surgery has expanded significantly in recent years, and new procedures continue to be developed that will continue that trend. The procedures are carried out with laparoscopic surgical instruments introduced through small, surgically created openings (ports) in a patient, such as illustrated in FIG. 1. A tubular device known as trocar (101) is placed in each opening. Typically, the trocar has a diameter of about 10-12 mm. An endoscope (102) with a miniature video camera is inserted through one of the trocars to provide a view of the surgical field (103) within. A laparoscopic surgical instrument (104) is then introduced through a given trocar, as depicted in FIG. 1. For example, an instrument with scissors blades may be inserted, used, and withdrawn. Next, another type of instrument, perhaps one to cauterize tissue, is inserted in the trocar, used and withdrawn. Then yet another type of instrument, perhaps one to provide suction, is introduced, used and withdrawn, and so forth. As a result, there are many exchanges of different types of instruments during a surgery, each of which must be withdrawn before another may be inserted and used, thereby not only extending the time of a surgical procedure but also increasing the chance of damage to the surrounding tissue due to human error.

A typical laparoscopic instrument (201) with scissors (202) consists of a long, hollow tube (203) of stainless steel, as illustrated in FIG. 2. The tube outer diameter is quite small (often 5 mm). Inside the tube is a stainless steel push-pull rod (204) that can be moved forward or backwards several millimeters along the longitudinal axis of the instrument. The motion is imparted via a mechanism attached to finger grips (205) that resemble those on conventional surgical scissors. At the other end of the instrument is a linkage (206) that converts the longitudinal motion of the push-pull rod into opening or closing of scissors blades. In addition, there is a knob (207) attached to the tube that can be turned to rotate the tube about its longitudinal axis, thereby allowing the angular orientation of the blades to be varied. The length of the tube and tool of such instruments are typically about 30 mm.

Other laparoscopic instruments have features similar to those in FIG. 2, except with different tools in place of scissors.

An internet-based literature search (PubMed and EI-Compendex) and a patent search (world wide web US Patent and Trademark Office Site) were conducted using keyword combinations such as “multifunctional+surgical+instrument+laparoscopic or endoscopic.” The search yielded dozens of patents and a number of journal articles. Copies of journal articles and patents that seemed relevant based on their titles and abstracts were obtained and reviewed. First, the articles thought to be most relevant in terms of presenting possible competing approaches are discussed.

Wallweiner et al. ((1995) “Multifunctional Instrument for Operative Laparoscopy” Endoscopic Surgery & Allied Technologies, 3(2-3): 119-124) describe a multifunctional instrument consisting of a 10 mm diameter “rod” attached to handle having a small keyboard. As depicted in FIG. 3, the rod (300) contains a needle electrode (301) for electrothermal cutting that can be advanced or retracted pneumatically, a 2.7 mm diameter channel (302) for suction or irrigation, and an open 5 mm diameter channel (303) into which various 5 mm diameter, conventional single purpose laparoscopic instruments can be inserted. The instrument was used in a number of different surgical procedures on pigs, reducing the length of procedures from 18 to 26%, depending on the procedure. Later, it was used successfully in clinical procedures. The main drawbacks to the instrument appear to be: (a) the need to manually insert and withdraw instruments through the open channel (303), (b) a “large” (10 mm) diameter in the surgical field, (c) the complexities and extra weight associated with pneumatic actuation, and (d) the inability to replace the needle electrode with another type of tool if desired.

A related type of multifunctional instrument is described by Farin ((1993) “Pneumatically Controlled Bipolar Cutting Instruments” Endosocopic Surgery & Allied Technologies, 1(2): 97-101). It consists of separate suction and irrigation channels within a large diameter “shaft.” Inside the irrigation channel is a needle electrode capable of being advanced or retracted pneumatically. This instrument appears to have drawbacks similar to those just described above.

Bakshi et al. ((2003) “A Multifunctional, Modified Rigid Neuroendoscopic System: Clinical Experience with 83 Procedures,” J. Neurosurgery, 99: 421-425) describe a multifunctional instrument designed specifically for neurosurgery. As illustrated in FIG. 4A and FIG. 4B, it consists of a 4 mm diameter telescope (180 mm long) (304), a 1 mm diameter insulated wire (305) leading to an electrode (306) for mono-polar dissection and coagulation electrode, and a small diameter (16 French) irrigation and suction tube (307), all bundled together with pieces of shunt tubing (308). That assembly is then placed inside a rigid 7.5 mm diameter tube (309), as illustrated in FIG. 4B. An additional small tool such as a catheter can be introduced into the remaining open space in the outer tube (310). This improvised instrument has been used successfully in clinical applications, but has a number of drawbacks. For example, to change the angular orientation of the electrode, the entire bundled assembly has to be withdrawn, rotated and then re-inserted. In addition, the authors note that it is sometimes difficult to maneuver an additional tool within the outer tube because of the presence of the bundle of other components. The instrument also lacks the versatility to accommodate scissors or a number of other types of tools.

Kunz (US Publication No. US 2004/0249366 A1, claiming priority to International Patent Application No. PCT/DE02/03581, filed 24 Sep., 2002, published 3 Apr. 2003) discloses a multifunctional instrument for use in microinvasive surgery that includes an operator hand grip, a multi-lumen tube fastened on the operator hand grip, at least two guide channels coaxially configured inside the tube, surgical instruments that are displaceably and rotationally disposed in the guide channels, a shaft at whose distal end one surgical working element each is disposed. The instruments can be displaced between a rest position, in which the respective working element is retracted into the tube, and a working position, in which the respective working element projects from the distal end of the tube. The instruments can be displaced into and out of the working position by means of a motor and at least one of the instruments can be rotated in its working position by means of a motor. The instrument can be controlled by means of an electronic control that controls at least the motor-driven displacement motions and rotational motions of the surgical instruments and the tube. The surgical instruments as disclosed displace back and forth in a rigid manner. The surgical instruments are built into the device, limiting its versatility. Kunz does not disclose that the multi-lumen tube can comprise a tapered portion.

There are several problems associated with exchanging laparoscopic instruments during a surgical procedure. First of all, it is time consuming and lengthens operating time, which, in turn, increases costs and the time a patient is anesthetized. Second, it may disrupt the flow of a surgical procedure and break a surgeon's concentration (Mehta et al., (2002) “Sequence and Task Analysis of Instrument Use in Common Laparoscopic Procedures” Surgical Endoscopy, 16: 280-285). Third, it results in loss of visual contact with the operating field (Waliweiner et al., (1995) supra). Such “blind episodes” can run the risk of injury to a patient. Also, because exchanging instruments can present such problems, a surgeon may make do with an instrument already “in place” within a patient, even though a more suitable instrument is available nearby. There is a need for a laparoscopic instrument that can provide multiple functions and thereby reduce the number of instrument exchanges needed. That capability could significantly reduce the length of a surgery, which would benefit a surgeon and his or her team, and produce substantial cost savings. Patients would also benefit from shortened surgeries and fewer risks associated with instrument exchanges.

### SUMMARY OF THE INVENTION

The invention is drawn to a surgical instrument having multiple tools. The instrument may provide at least two functions, which may include, but is not limited to, suction, irrigation, irradiation, dissection; cauterization, adhesion, suturing, or the like, via a single distal end opening. In a preferred embodiment the distal end opening is tapered. In another preferred embodiment the instrument is adapted for single-handed operation and can be used by an operator using only one hand.

In one embodiment the invention provides a multi-functional surgical instrument, the instrument comprising a body and a plurality of tools, the body defining a lumen and the tools disposed therewithin. In a preferred embodiment the body of the instrument comprises a tapered distal portion, the tapered distal portion comprising at least one percent of the length of the body and wherein the tapered distal portion has a distal end and a proximal end. In a more preferred embodiment the tapered distal portion comprises at least two percent of its length. In a still more preferred embodiment the tapered distal portion comprises at least five percent of its length. In a yet more preferred embodiment the tapered distal portion comprises at least ten percent of its length. In a preferred embodiment the distal end has a cross-sectional area selected from the group consisting of about one half, about one third, about one quarter, about one fifth, about one sixth, about one eighth, and about one tenth, of that of the cross-sectional area of the proximal end. In a more preferred embodiment the distal end has a cross-sectional area equal to about one quarter that of the cross-sectional area of the proximal end. In an alternative embodiment the tapered distal portion comprises a separate item of manufacture and may be shaped and adapted for removable assembly with the body. The tapered distal portion can comprise a rigid material that resists deformation along its structure thereby exerting positive pressure upon a tool disposed therewithin, the tool thereby becoming reversibly deformed along its length. The tapered distal portion can optionally comprise ribs disposed logitudinally along the tapered distal portion, wherein the ribs may comprise a stiff and/or rigid material and the material therebetween may comprise an elastomeric and/or stretchable material. In one preferred embodiment, the tool flexes only when advanced or retracted through the first lumen. In one alternative embodiment, the tapered distal portion comprises different materials, the different materials having different physical properties, such as elastomers, rigid materials, or materials having properties therebetween. In a preferred alternative embodiment, the tapered distal portion comprises a material that is substantially flexible to accommodate tools having different shapes and sizes.

In a preferred embodiment, the body and all other components comprise a composition selected from the group consisting of stainless steel, copper, aluminum, titanium, metal matrix composite, DELRIN (acetal), acrylonitrile butadiene styrene (ABS), nylon, polypropylene, polybromate, polycarbonate, glycolised polyethylene terephthalate (PETg) copolyester, glass fiber-resin composites, other composite materials, and the like.

In another embodiment the multi-functional surgical instrument as disclosed above further comprises a tool, wherein the tool has a distal portion and a proximal portion and comprises a flexible material. In a preferred embodiment the tool flexes over at least one percent of its length. In a more preferred embodiment the tool flexes over at least two percent of its length. In a still more preferred embodiment the tool flexes over at least five percent of its length. In a yet more preferred embodiment the tool flexes over at least ten percent of its length. In another preferred embodiment the tool flexes at the distal portion of the tool. In a still further preferred embodiment the tool flexes at an angle of arc of at least one degree and wherein the angle of arc is measured between a point on the distal portion of the tool and a point on the proximal portion of the tool. The tool can have a length of between about 5 cm and about 150 cm. In particular the tool can have a length, for example, of about 5 cm, 6 cm, 7 cm, 8 cm, 9 cm, 10 cm, 12.5 cm, 15 cm, 20 cm, 25 cm, 30 cm, 40 cm, 50 cm, 60 cm, 70 cm, 80 cm, 90 cm, 100 cm, 125 cm, and 150 cm.

In one embodiment the tool is shaped and adapted for advancing and retracting within the lumen of the body. In a preferred embodiment the tool is shaped and adapted for advancing, retracting, and rotating within the lumen of the body. In another preferred embodiment, the tool is adapted for use with an electric operating system. In a still further embodiment the tool is adapted for passage of fluids. In a preferred embodiment, the adaptation for passage of fluids comprises tubing selected from the group consisting of irrigation tubing and suction tubing. In another preferred embodiment, the tools are selected from the group consisting of disposable tools and re-usable tools. In one more preferred embodiment, the tools are disposable. In an alternative preferred embodiment, the tools are re-usable. In another preferred embodiment, the multifunctional surgical instrument comprises tools having different dimensions. In another preferred embodiment the tool is adapted for passage of solids, wherein the solids are selected from the group consisting of tissue debris, blood clots, and lymph material, and the adaptation for passage of solids comprises tubing selected from the group consisting of irrigation tubing and suction tubing.

In an alternative embodiment, the body comprises a distal end and a proximal end, the distal end having a cross-sectional area equal to that of the proximal end.

In a preferred embodiment the multifunctional surgical instrument is adapted for use with one hand. In another preferred embodiment the multifunctional surgical instrument is adapted for use with two hands.

In certain alternative embodiments, the body of the instrument is a hollow first tube or sheath having a distal end and a proximal end, the two ends defining a first lumen and designed to receive at least one tool within the first lumen. The tool has a distal end and a proximal end. The tool is fixedly attached to a second tube, the second tube having a distal end and a proximal end and having a second lumen defined by the distal end and the proximal end of the second tube. In a preferred embodiment, the proximal end of the tool is attached to the distal end of the second tube. The instrument is designed such that, in use, the distal end of the tool is positioned within the first lumen of the first tube at or near the distal tip of the body of the instrument. The proximal end of the second tube further comprises a knob with which an operator can hold and thereby allows the operator to slide the tool freely back and forth within the first lumen of the first tube. In a preferred embodiment, the knob is fixedly attached to the second tube. In the alternative, the knob is attached to the second tube using interconnecting means, the interconnecting means selected from the group consisting of retaining rings, clips, and the like. The interconnecting means can allow the second tube to rotate relative to the knob. In another embodiment, the knob further comprises a rod, the rod fixedly attached to the second tube using interconnecting means and the rod extending laterally from and perpendicular to the second tube.

In an alternative embodiment, the instrument further comprises an electron-conducting material removably attached to the proximal end of the second tube. In a preferred embodiment, the electron-conducting material is partially encased in an electron-insulating material.

The distal end of the instrument comprises an aperture through which the tool can be positioned therethrough. The tool can be repositioned within the first lumen by the operator, the operator gripping the knob and sliding the tool and the second tube through the first lumen towards the distal tip of the body of the instrument resulting in the distal end of the tool emerging through the distal tip of the body of the instrument.

In one preferred embodiment, the instrument comprises multiple tools, the tools selected from the group consisting of, scissors, forceps, spoon forceps, graspers, a clip, clip applier, clip applicator, dissector, stapler, hook, laser-emitting device or laser delivery system, an obturator, cauterizing wire, electrocautery devices including monopolar and bipolar instruments, glue gun, suction tubing, pressure tubing, irrigation tubing, and the like. In a more preferred embodiment, the instrument comprises different tools, the tools having been pre-selected for use by the operator for a particular surgical procedure. In a preferred embodiment, the instrument comprises a second tube having a diameter smaller than that of the first tube thereby allowing the tool to move freely within the first lumen of the first tube. The cross-section of the second tube can be selected from the group consisting of round, square, oblong, triangular, a pentagon, a hexagon, an octagon, a polygon, similar to the sector of a circle, and/or it can be designed and manufactured such that multiple tools may be positioned and stored within the lumen of the first tube in a more space-saving manner. In a preferred embodiment the tools may be introduced and removed from the lumen of the instrument without the removal of the instrument from a patient's body. In another preferred embodiment, the tools or modules are the same tools or modules. In another preferred embodiment, the tools or modules have different dimensions. In a still further preferred embodiment, the tools or modules are used simultaneously.

The distal end of the first tube has a diameter less than the diameter of the proximal end of the first tube, the distal portion of the first tube tapering from a point at between about 1% and about 100% of the length of the tube measured from the proximal end. The taper can be, for example, from about 1%, 2%, 3%, 4%, 5%, 7.5%, 10%, 15%, 20%, 30%, 40%, 50%, 60%, 70%, 80%, 90%, and 100% of the length of the tube measured from the proximal end. The diameter of the distal end can be between about 1 mm to about 50 mm or for example, about 1 mm, 1.5 mm, 2 mm, 2.5 mm, 3 mm, 4 mm, 5 mm, 6 mm, 7 mm 8 mm 9 mm, 10 mm, 11 mm, 12 mm, 13 mm, 14 mm, 15 mm, 18 mm, 20 mm, 25 mm, 30 mm, 40 mm, or 50 mm in diameter. The taper does not compromise the function of the tool. The purpose of the taper is to facilitate surgical maneuvering and to provide a good view of the surgical field.

The second tube of the tool is adapted and manufactured to be rigid but flexible. The second tube can also be a rod, for example, comprising a solid composition and/or material shaped as a cylinder. The second tube comprises a composition selected from the group consisting of stainless steel, copper, aluminum, titanium, metal matrix composite, DELRIN (acetal), acrylonitrile butadiene styrene (ABS), nylon, polypropylene, polybromate, polycarbonate, glycolised polyethylene terephthalate (PETg) copolyester, glass fiber-resin composites, other composite materials, and the like. In one embodiment the second tube is adapted to conduct an electric current. In another embodiment the second tube is shaped and adapted to allow a plurality of second tubes to be packed and stored within the first lumen of the first tube. In an alternative embodiment the second tube can further comprise a push-pull rod, shaped and adapted for positioning within the second lumen of the second tube. The push-pull rod is shaped and adapted to avoid friction resulting in contact with the inner surface of the second tube and to flex. In another alternative embodiment the push-pull rod is adapted to conduct an electric current.

In another embodiment, the body of the instrument further comprises a casing, the casing fashioned from a rigid material, such as a metal or a polymeric compound. The casing has a distal end and a proximal end. In one embodiment the cross-section of the casing can be selected from the group consisting of round, square, oblong, triangular, a pentagon, a hexagon an octagon, a polygon, and similar to the sector of a circle. The casing can have four sides or less. The casing has the form of a tube, having an inner surface and an outer surface. The distal end and the proximal end and the inner surface define a third lumen. In a preferred embodiment, the first tube is longitudinally disposed within the third lumen of the casing. The casing further comprises a plurality of slots, each slot disposed longitudinally along the length of the casing, and the slot connecting the outer surface with the inner surface of the casing. At least one slot is disposed upon one side of the casing. Another slot can be disposed upon the opposing side of the casing. The slot is shaped and adapted to receive the interconnecting means of the second tube and the knob. In another embodiment the slot is shaped and adapted to receive the rod.

In another embodiment, the casing comprises a first support member, the first support member shaped and adapted for supporting the first tube in the casing. The first support member further comprises a first aperture, the aperture shaped and adapted to receive the first tube therethrough. In another embodiment, the first support member further comprises a second aperture, the second aperture shaped and adapted to receive the second tube therethrough.

In another embodiment, the casing further comprises a second support member, the second support member shaped and adapted for supporting the first tube in the casing. In an additional embodiment the second support member shaped and adapted for supporting the second tube in the casing. In another embodiment the support member further comprise a bracket, the bracket comprising two distal ends and further comprising a first pin disposed between the distal ends of the bracket. In one embodiment the first pin is partly disposed within an aperture located near each of the distal ends of the bracket.

In another embodiment, the instrument comprises first compression and expansion means, the compression and expansion means disposed between the support member and the interconnecting means of the knob. The compression and expansion means can be selected from the group consisting of a spring, an inflatable bag or balloon, a hydraulic system, and the like.

In another embodiment, the casing further comprises a pair of third support members, the support members shaped and adapted for providing a barrier that impedes movement of the second tube beyond a predetermined position within the casing. The third support members further comprise at least one third aperture each, the third aperture shaped and adapted for receiving the first pin. In a preferred embodiment the third support members further comprise a fourth aperture each, the fourth aperture shaped and adapted for receiving a second pin. In another embodiment the third support members further comprise a notch, shaped and adapted for receiving the rod.

In another embodiment the body can comprise at least two casings, the two casings having different cross-sectional dimensions. This can allow an operator to use at least two tools having different diameters and/or dimensions from one another. This can be an advantage when multiple tools are necessary for a surgical procedure.

In another embodiment, the instrument comprises a first handle, the first handle fixedly attached to the third support members.

In a preferred embodiment, the instrument further comprises a pair of gears, the gears shaped and adapted for rotating against one another. Both gears are shaped and adapted to intermesh with each other when brought into contact. In one preferred embodiment the gears are tapered gears, wherein the gears are shaped and adapted to engage with one another whilst the tool is rotating.

In one embodiment the ratio of the first gear to the second gear is selected from the group consisting of 2:1, 3:1, 4:1, 5:1, 6:1, 7:1, 8:1, 9:1, 10:1, and intermediate values thereof. In a preferred embodiment, the pair of gears have a ratio of about 4:1. In a preferred embodiment the smaller gear is fixedly attached to the outer surface of the second tube. In a preferred embodiment, the first gear is fixedly attached to an axle, the axle mounted upon the casing passing through the second aperture. In a preferred embodiment the axle further comprises a second handle, the shaft of the second handle extending distally from and perpendicular to the axel. The axle further comprises second compression and expansion means that restricts longitudinal movement of the first gear relative to the second gear, the means disposed upon or adjacent to the outer surface of the axle. In a preferred embodiment the gears are shaped and adapted to engage with one another whilst the tool is rotating.

In another embodiment, the casing comprises an end plate, the end plate disposed at the proximal end of the casing and shaped and adapted for receiving and supporting the proximal end of the second tube. In an alternative embodiment, the end plate is shaped and adapted for receiving and supporting the electron-conduction material.

In a yet further embodiment of the invention, the instrument comprises a grip, the grip shaped and adapted for receiving the hand of an operator. It can be shaped for use in the left hand, it can be shaped for use in the right hand, or it can be shaped for use in the left hand or the right hand.

In another embodiment, the instrument comprises at least one flexible tube, adapted for transmitting fluids. In another embodiment the instrument comprises at least one valve button for regulating the flow of fluid. In a preferred embodiment, the valve button is disposed upon the grip. In one embodiment the fluid is an aqueous solution that may be used for irrigation. In an alternative embodiment the fluid is a gas. In another alternative embodiment the flexible tube is in fluid communication with a vacuum pump and the tube can be used for suction.

In a still further embodiment the invention provides for a multifunctional surgical instrument having at least one tool, the instrument having a single distal aperture and a single proximal aperture, wherein the instrument is tapered between the single distal aperture and the single proximal aperture thereby providing a tapered distal end portion and wherein the tool, in use, reversibly protrudes from the single distal end. In an alternative embodiment the multifunctional surgical instrument has at least two tools, the instrument having a single distal aperture and a single proximal aperture, wherein the instrument is tapered between the single distal aperture and the single proximal aperture thereby providing a tapered distal end portion and wherein the tools, in use, reversibly protrude from the single distal end. In one preferred embodiment, the multifunctional surgical instrument further comprised means adapted for passage of fluids, wherein the means adapted for passage of fluids comprises tubing selected from the group consisting of irrigation tubing and suction tubing. In another alternative embodiment, the multifunctional surgical instrument further comprises means adapted for passage of solids wherein the solids are selected from the group consisting of tissue debris, blood clots, and lymph material and the adaptation for passage of solids comprises tubing selected from the group consisting of irrigation tubing and suction tubing. In a still further preferred embodiment the multifunctional surgical instrument further comprises a pair of tapered gears, wherein the gears are shaped and adapted to engage with one another whilst the tool is rotating.

In an alternative preferred embodiment, the invention provides a multifunctional surgical instrument, the surgical instrument comprising: a first tube, the first tube having a proximal end and a distal end, the two ends defining a first lumen, wherein a distal portion of the first tube is tapered and wherein the distal end of first tube has a cross-sectional area of about one quarter the cross-sectional area of the proximal end of the first tube, a casing, the casing having a proximal end and a distal end, the proximal end and the distal end defining a lumen of the casing, the casing further comprising at least two first pins, the first pin disposed upon the side of the casing, a first operator hand grip, the first hand grip being attached to a portion of the casing, at least two second tubes, the at least two second tubes each having a proximal end and a distal end, the two ends defining a second lumen and the second tubes being disposed within the first lumen, a tool attached to the distal end of the second tube, the tool having a proximal end and a distal end, the tool further comprising a first rod, the first rod comprising a proximal end and a distal end, the distal end being operably connected with the tool and the proximal end being operably connected with a link, the link being operably connected with a knob, and wherein the first rod further comprises a first gear, the first gear comprising a solid conic section, the solid conic section defining the surface of the first gear and further comprising cogs arranged upon the curved surface of the conic section, a second gear, the second gear rotatably mounted upon the casing, the second gear further comprising a solid conic section, the solid conic section defining the surface of the second gear and further comprising cogs arranged upon the curved surface of the solid conic section, wherein the ratio of the first gear to the second gear is at least 1:2, a compression and expansion means, the compression and expansion means comprising a proximal end and a distal end, the compression and expansion means being disposed longitudinally within the lumen of the casing parallel to the surface of the second tube and the first rod and wherein the distal end of the compression and expansion means abuts the second plate and the proximal end of the compression and expansion means abuts the link, and a second operator hand grip, the second hand grip operably attached to the pair of third plates.

In the alternative, the invention provides a multifunctional surgical instrument, the surgical instrument comprising: a first tube, the first tube having a proximal end and a distal end, the two ends defining a first lumen, wherein a distal portion of the first tube is tapered and wherein the distal end of first tube has a cross-sectional area of about one quarter the cross-sectional area of the proximal end of the first tube, a casing, the casing having a proximal end and a distal end, the proximal end and the distal end defining a lumen of the casing, the casing further comprising at least two first pins, the first pin disposed upon the side of the casing, a first operator hand grip, the first hand grip being attached to a portion of the casing, at least two second tubes, the at least two second tubes each having a proximal end and a distal end, the two ends defining a second lumen and the second tubes being disposed within the first lumen, a tool attached to the distal end of the second tube, the tool having a proximal end and a distal end, the tool further comprising a first rod, the first rod comprising a proximal end and a distal end, the distal end being operably connected with the tool and the proximal end being operably connected with a link, the link being operably connected with a knob, and wherein the first rod further comprises a second pin and a first gear, the first gear comprising a solid conic section, the solid conic section defining the surface of the first gear and further comprising cogs arranged upon the curved surface of the conic section, a second gear, the second gear rotatably mounted upon the casing, the second gear further comprising a solid conic section, the solid conic section defining the surface of the second gear and further comprising cogs arranged upon the curved surface of the solid conic section, wherein the ratio of the first gear to the second gear is at least 1:2, a first plate, the first plate disposed at the junction of the first tube and the casing and wherein the first plate has at least two first apertures through which the at least two second tubes are disposed, a second plate, the second plate attached to the casing, a first bracket, the first bracket attached to the second plate and perpendicular to the second plate, the first bracket further comprising a second pin, a compression and expansion means, the compression and expansion means comprising a proximal end and a distal end, the compression and expansion means being disposed longitudinally within the lumen of the casing parallel to the surface of the second tube and the first rod and wherein the distal end of the compression and expansion means abuts the second plate and the proximal end of the compression and expansion means abuts the link, a pair of third plates, wherein the two third plates are attached to each other with a second connection rod therebetween allowing the third plates to rotate freely about the second connection rod, wherein each third plate further comprises a first slot, a second slot, and a second aperture, wherein each third plate is shaped and adapted to reversibly receive and engage the second pin within the first slot and immobilize the second pin therein, and wherein the second aperture of each third plate is shaped and adapted to engage the second pin, and wherein the second slot of each third plate is shaped and adapted to reversibly receive and engage the first pin within the second slot and immobilize the first pin therein, and a second operator hand grip, the second hand grip operably attached to the pair of third plates.

In another alternative, the invention provides a multifunctional surgical instrument, the surgical instrument comprising: a first tube, the first tube having a proximal end and a distal end, the two ends defining a first lumen, wherein a distal portion of the first tube is tapered and wherein the distal end of first tube has a cross-sectional area of about one quarter the cross-sectional area of the proximal end of the first tube, a casing, the casing having a proximal end and a distal end, the proximal end and the distal end defining a lumen of the casing, a first operator hand grip, the first hand grip being attached to a portion of the casing, at least two second tubes, the at least two second tubes each having a proximal end and a distal end, the two ends defining a second lumen and the second tubes being disposed within the first lumen, wherein the proximal end is operably connected with a link, the link being operably connected with a knob, and wherein the second tube further comprises a first gear, the first gear comprising a solid conic section, the solid conic section defining the surface of the first gear and further comprising cogs arranged upon the curved surface of the conic section, a second gear, the second gear rotatably mounted upon the casing, the second gear further comprising a solid conic section, the solid conic section defining the surface of the second gear and further comprising cogs arranged upon the curved surface of the solid conic section, wherein the ratio of the first gear to the second gear is at least 1:2, a tool attached to the distal end of the second tube, the tool having a proximal end and a distal end, the tool further comprising a first rod, the first rod comprising a proximal end and a distal end, the distal end being operably connected with the tool, wherein the rod is disposed within the second lumen, a compression and expansion means, the compression and expansion means comprising a proximal end and a distal end, the compression and expansion means being disposed circumpherentially upon the surface of the second tube and wherein the distal end of the compression and expansion means abuts the first plate and the proximal end of the compression and expansion means abuts the first gear, and a second operator hand grip, the second hand grip operably attached to the pair of third plates.

In a yet further alternative, the invention provides a multifunctional surgical instrument, the surgical instrument comprising: a first tube, the first tube having a proximal end and a distal end, the two ends defining a first lumen, wherein a distal portion of the first tube is tapered and wherein the distal end of first tube has a cross-sectional area of about one quarter the cross-sectional area of the proximal end of the first tube, a casing, the casing having a proximal end and a distal end, the proximal end and the distal end defining a lumen of the casing, a first operator hand grip, the first hand grip being attached to a portion of the casing, at least two second tubes, the at least two second tubes each having a proximal end and a distal end, the two ends defining a second lumen and the second tubes being disposed within the first lumen, wherein the proximal end is operably connected with a link, the link being operably connected with a knob, and wherein the second tube further comprises a first gear, the first gear comprising a solid conic section, the solid conic section defining the surface of the first gear and further comprising cogs arranged upon the curved surface of the conic section, a second gear, the second gear rotatably mounted upon the casing, the second gear further comprising a solid conic section, the solid conic section defining the surface of the second gear and further comprising cogs arranged upon the curved surface of the solid conic section, wherein the ratio of the first gear to the second gear is at least 1:2, a tool attached to the distal end of the second tube, the tool having a proximal end and a distal end, the tool further comprising a first rod, the first rod comprising a proximal end and a distal end, the distal end being operably connected with the tool, wherein the rod is disposed within the second lumen, a first plate, the first plate disposed at the junction of the first tube and the casing and wherein the first plate has at least two first apertures through which the at least two second tubes are disposed, a first bracket, the first bracket attached using a second pin to a second bracket, the second bracket positioned parallel to three sides of the casing, the first bracket further comprising at least two third pins, the second bracket comprising a fourth pin and a fifth pin, wherein the fifth pin attaches the second bracket to the casing, wherein the two brackets are movably attached to each other with a sixth pin therebetween allowing the two brackets to rotate freely about the sixth pin, wherein the first bracket further comprises a ratchet, wherein ratchet is shaped and adapted to reversibly receive and engage the first pin within the first slot and immobilize the first pin therein, a torsion spring, the torsion spring attached to the at least two third pins and the fourth pin, thereby creating a torsion force, a compression and expansion means, the compression and expansion means comprising a proximal end and a distal end, the compression and expansion means being disposed circumpherentially upon the surface of the second tube and wherein the distal end of the compression and expansion means abuts the first plate and the proximal end of the compression and expansion means abuts the first gear, and a second operator hand grip, the second hand grip operably attached to the pair of third plates.

The invention also provides the instrument disclosed herein further comprising a plurality of modules comprising at least one tool. The module is shaped and adapted for positioning within the first lumen of the instrument. In a preferred embodiment the module is shaped and adapted for advancing and retracting within the first lumen. In another preferred embodiment the module is shaped and adapted for rotating about its longitudinal axis within the first lumen. In the alternative, the modules are shaped and adapted for attaching to the distal end of the instrument, the tool is shaped and adapted for reversible attachment to the distal end of the instrument, and/or the module is shaped and adapted for mechanical engagement with other components of the instrument. In another embodiment the tool is shaped and adapted for mechanical engagement with other components of the instrument.

The invention can be used in surgical procedures for any organism, such as humans, domesticated animals, wild animals, plants, and the like. Alternatively, the invention can be used in technical procedures, such as retrieving, manipulating, and positioning small objects in a large device wherein access to the small objects is limited due to space or entry points. The instrument can be used with a robotic device, the robotic device comprising a plurality of modules.

### BRIEF DESCRIPTION OF THE DRAWINGS

FIG. 1 illustrates how instruments are positioned during a laparoscopic procedure in current use.

FIG. 2 illustrates the typical components of a laparoscopic scissors instrument in current use.

FIG. 3 is a schematic of the working end of the multifunctional instrument of Wallweiner et al. ((1995) supra).

FIG. 4 illustrates (a) the components and (b) cross-sectional view of neuroendoscopic, multifunctional instrument Bakshi et al. ((2003) supra).

FIG. 5 illustrates an exemplary embodiment of the invention in a cross sectional view showing two tools retracted and one tool deployed.

FIGS. 6A, 6B, and 6C illustrate an exemplary embodiment of the instrument, showing the tools in different positions during deployment and use.

FIG. 7 illustrates another exemplary embodiment of the instrument in different cross-sectional illustrations. FIGS. 7A and 7B illustrate an exemplary distal portion of the instrument; FIGS. 7C and 7D illustrate an exemplary central portion of the instrument; FIGS. 7E and 7F illustrate an exemplary proximal portion of the instrument; FIG. 7G is a cross-sectional view of the proximal portion of the instrument shown in FIG. 7B; FIG. 7H is a cross-sectional view of the proximal portion of the instrument shown in FIG. 7C; FIG. 7I is a three-quarter view of the plate (52).

FIG. 8 is a detailed view of the distal tapering portion of the first tube illustrating means for replacement and removal of the distal end from the instrument.

FIG. 9 is a three quarter view of the proximal portion of the instrument and the grip.

FIG. 10 is a detailed view of the end of a second tube, its push-pull rod, and an electric attachment. FIG. 10B illustrates a cross-section of the electrical connections between the electrical elements and the push-pull rod.

FIG. 11 illustrates exemplary modules of the instrument; scissors module (FIG. 11A) and J-hook electrocautery module (FIG. 11B).

FIG. 12 illustrates an embodiment of a pair of gears of the invention.

FIG. 13 illustrates an alternative embodiment of the tapered portion of the first tube. FIG. 13B is a cross-sectional view of the tube.

FIG. 14 illustrates an alternative embodiment of the invention that may be operated with one hand.

FIG. 15 illustrates an exemplary alternative embodiment of a tool module.

FIG. 16 illustrates an exemplary location of the gears in an instrument that can be operated with one hand.

FIG. 17 illustrates an exemplary mechanism for using the invention.

FIG. 18 illustrates an exemplary embodiment of the invention in use. FIG. 18A is a view from above; FIG. 18B is a view from the side.

FIG. 19 illustrates an embodiment of the invention showing the relative positions of the push-pull rods and tools of the invention. FIG. 19A is a view from above; FIG. 19B is a view from the side.

FIG. 20 illustrates a part of the proximal portion of the invention. FIG. 20A is a view from above; FIG. 20B is a view from the side.

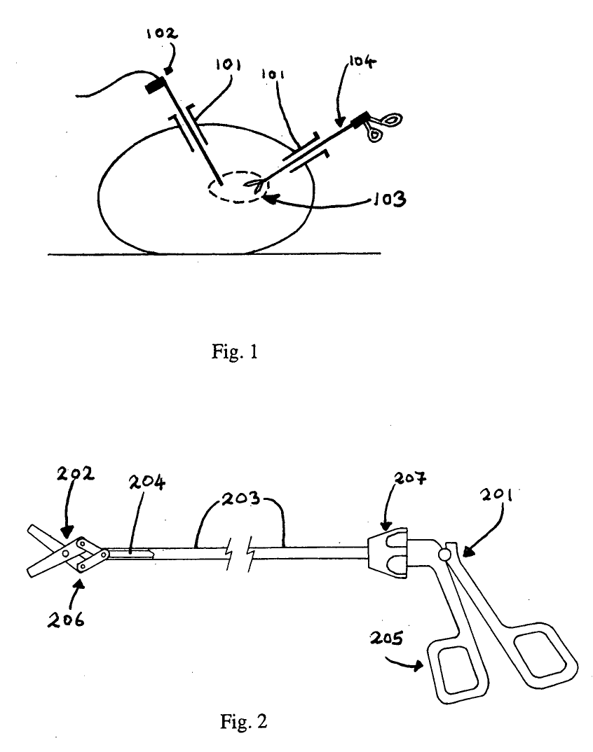

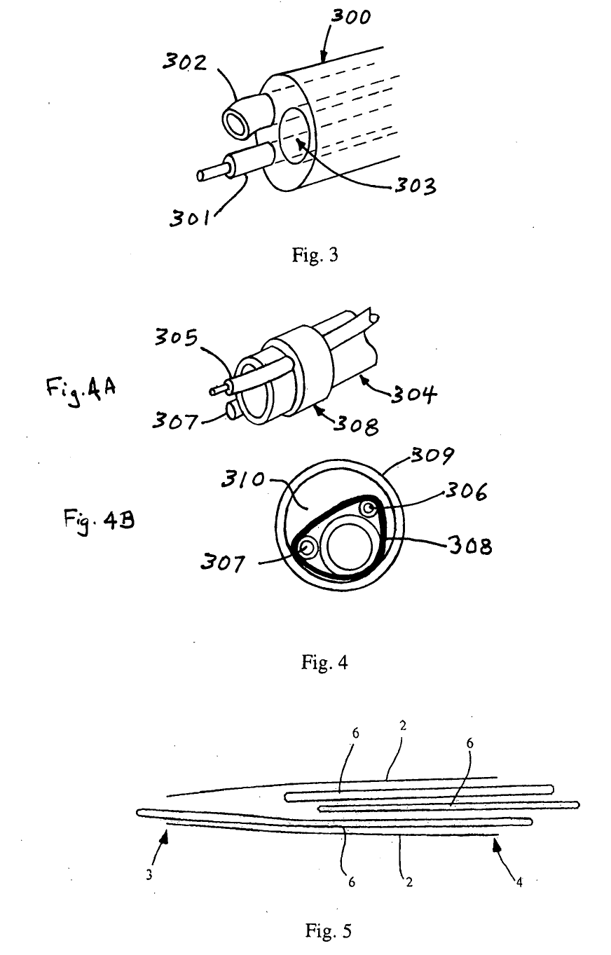

### DETAILED DESCRIPTION OF THE INVENTION

The embodiments disclosed in this document are illustrative and exemplary and are not meant to limit the invention. Other embodiments can be utilized and structural changes can be made without departing from the scope of the claims of the present invention.

As used herein and in the appended claims, the singular forms “a,” “an,” and “the” include plural reference unless the context clearly dictates otherwise. Thus, for example, a reference to “a rod” includes a plurality of such rods, and a reference to “a tool” is a reference to one or more tools and equivalents thereof, and so forth.

In the following, the term “instrument” denotes the entire device. The term “tool” can mean scissors, forceps, spoon forceps, graspers, cauterizing wire, glue gun, other devices that can perform a surgical function, light-emitting devices, lamps, radiant heat devices, heat transducers, measuring devices, and electrical coils. The tool can also comprise an elongate element, the elongate element comprising a flexible material. The term “module” refers to an assembly of tools, and an assembly of tools and other devices, such as for example, consisting of a tool plus an attached flexible tube and push-pull rod, as well as other mechanical devices attached to the tube or push-pull rod or the like. The term “module” can also refer to portions of the device that are manufactured for single or multiple use and that can be rapidly removed and attached to other portions of the instrument.

It should be noted that many details of the design described in the following are for the purpose of making a prototype for testing, and would be modified in a product to improve appearance, manufacturability, etc.

A primary purpose of the laparoscopic instrument described here is to reduce the frequency of instrument exchanges by enabling a number of commonly encountered surgical functions to be performed with one instrument.

A multifunctional laparoscopic instrument was conceived and designed with the following considerations in mind:
(a) It can provide scissors, electrocautery, suction, and/or irrigation, such functions as commonly used in various laparosocopic procedures. To enhance its versatility, the instrument can also be able to accommodate a variety of other combinations of tools in lieu of scissors and electrocautery tools.
(b) It can be possible to rapidly switch back and forth between functions such as scissors, an electrocautery device, suction and irrigation while the instrument remains in place during a procedure.
(c) The diameter of the tubing of the multifunctional instrument in the vicinity of the tool (for example, scissors) can be comparable to that in existing single purpose laparoscopic instruments to allow for fine surgical maneuvers. In particular, the portion of the instrument extending from a tool to a location about 8 to 10 cm closer to the grip can be small in diameter. The rest of the instrument could be up to 10 mm in diameter, enabling it to fit within commonly used 10 mm diameter trocars.
(d) Means should be provided to allow scissors or other tools to be rotated about their longitudinal axes so that they can be used in any desired angular orientation.
(e) The size and weight of the instrument should be minimized to the extent possible given its multifunctionality.
(f) The instrument may be robust, reliable and capable of being autoclaved repeatedly.
(g) The cost of the multifunctional instrument may be comparable to the sum of the costs of individual instruments, the functions of which are being incorporated into the instrument.

The present invention provides various advantages over the prior art devices and methods. The present invention provides instruments that comprise a plurality of tools, the instrument that can be positioned at a surgical site and that need not be removed by the operator during a surgical procedure. The invention further provides a tapered distal portion, an improvement over the prior art, that enables an operator to direct a surgical procedure within a smaller confine using multiple tools in one instrument compared with using an instrument having a larger distal portion that accommodates multiple tools.

One advantage of the instant invention is that the tool or module can bend while being advanced or retracted within the tapered portion of the lumen of the first, tube and maintain its function. The bending or flexing of the tool or module as it is being deployed or retracted in the tapered portion of the first tube is advantageous in that (a) it takes advantage of the flexibility of the tool or module in a novel way and (b) the tapered portion provides a better view of the surgical filed of view compared with that of a first tube having a constant diameter, thereby facilitating surgical maneuvering and surgical procedures. This advantage also applies to the second tube and the push-pull rod as disclosed herein.

Another advantage is that the tool may be rotated within the lumen of the instrument. This advantage is compounded inasmuch that the tool can be retracted and deployed at the same time that it is being rotated, thereby adding additional manipulative and surgical techniques available to the operator.

A further advantage of such an instrument is that the tapered distal portion (tapered tube) can reduce the footprint of the instrument in use both within the area or cavity subject to surgery or other clinical manipulations and within the field of view of an operator.

An additional advantage of such a instrument is decreased time spent performing surgical procedures. In the past, different laparoscopic instruments would be used for different tasks and procedures. Each instrument would be inserted, the position of the distal end of the instrument would be observed using an endoscope and viewing instrument, the instrument would be manipulated and adjusted to the required position, the instrument would be used in the surgical procedure, the instrument then withdrawn, and another laparoscopic instrument inserted, positioned, used, and withdrawn in turn. The present invention overcomes the procedures of repeatedly withdrawing, inserting, and re-positioning a laparoscopic instrument.

This last advantage is particularly important as removal and reinsertion of a instrument slows surgical procedures, may increase trauma, and can significantly impact surgical outcomes. Additionally, the present invention is simple and inexpensive to manufacture, simple to use and robust in use, and can be used with a variety of surgical instruments and tools.

FIG. 5 illustrates an embodiment of the invention showing how multiple tools (6) may be used with the invention. The figure is a cross-section of one exemplary embodiment. The instrument comprises a first tube (2) having a tapered distal portion (3) and a more proximal portion (4) having a diameter suitable for enclosing multiple tools (6). One of the tools (6) is shown in use extending and deployed from the distal end of the first tube wherein the tool (6) flexes along a portion of its length. The flexing is caused by the advancing the tool towards the distal portion where the tool encounters the tapered portion and is therefore forced by the internal walls of the first tube to accommodate itself to the change in direction. A similar tool comprising a more rigid material would not flex and therefore would be unlikely to be extendable from the distal end of the first tube (2). Other tools (6) are shown within the lumen of the first tube and are not deployed.

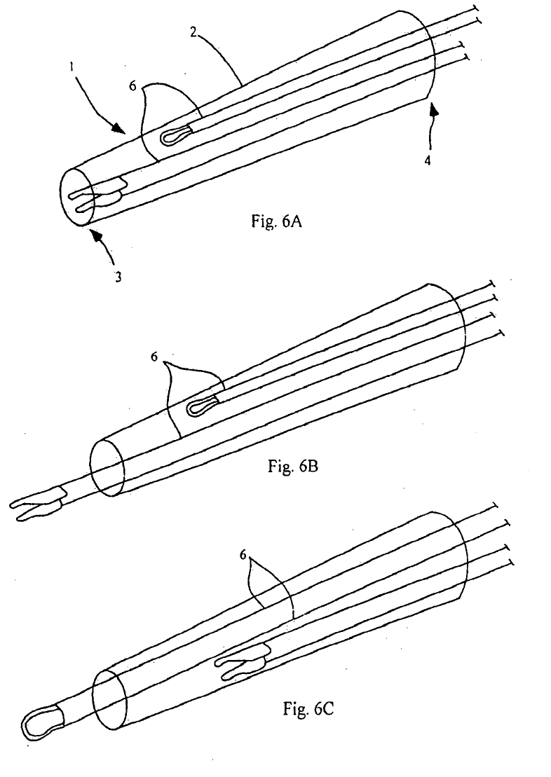

FIGS. 6A, 6B, and 6C show a series of illustrations that disclose how two different tools can be used in sequence. The instrument (1) comprises a first tube (2) having a tapered distal portion (3) and a more proximal portion (4) having a diameter suitable for enclosing multiple tools (6). FIG. 6A shows a scissors tool at the distal end of the first tube and a cauterizing wire tool stored in the lumen of the first tube. FIG. 6B shows the scissors tool having been extended from the distal end of the first tube and awaiting surgical use and the cauterizing wire tool remaining stored in the lumen of the first tube. FIG. 6C shows the cauterizing wire tool having been extended from the distal end of the first tube and awaiting surgical use and the scissors tool retracted and thereby remaining stored in the lumen of the first tube.

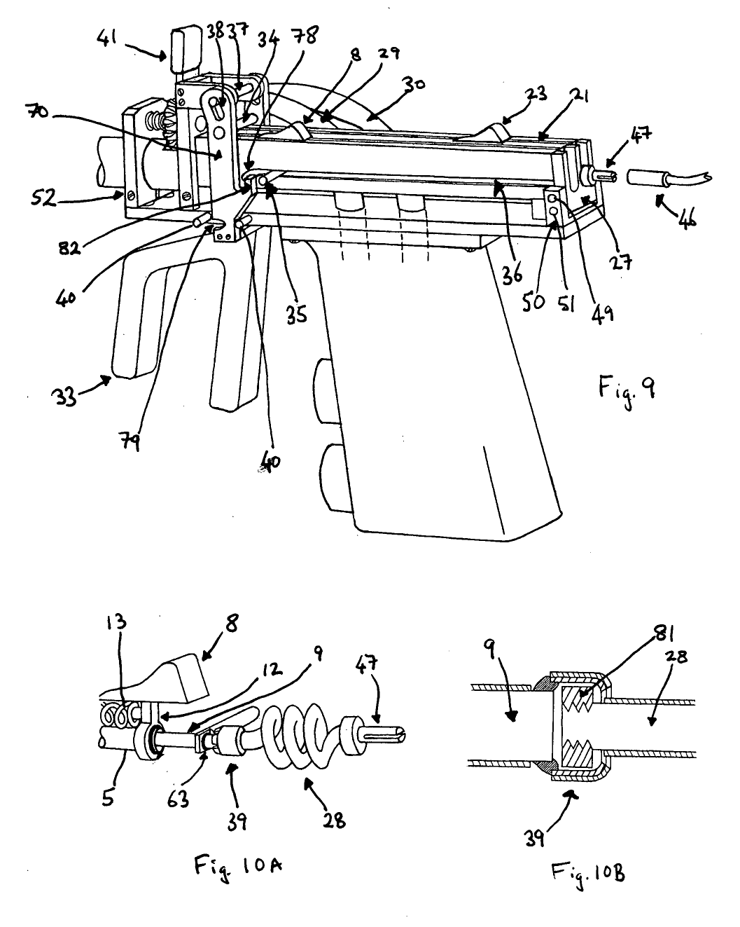

FIG. 7 provides an illustration of one exemplary embodiment of the instrument. Two views of the distal portion of the instrument are illustrated: a top view (FIG. 7A), and a side view (FIG. 7B), showing the components inside the first tube viewed from the side. FIG. 9 is a three quarter view of the portion of the instrument near the grip. FIGS. 10A and 10B are a detailed view of the end of a second tube and of the push-pull rod.

As depicted in FIGS. 7A and 7B, the instrument (1) comprises a first tube (2) of about 10 mm diameter wherein the distal portion of the tube tapers over a length of about 8 cm to a diameter of about 5 mm at the distal end (3) of the instrument. The reason for the taper is discussed below. The distal end of the first tube has a cross-sectional area selected from the group consisting of about one half, about one third, about one quarter, about one fifth, about one sixth, about one eighth, about one tenth, and any intermediate values thereof, to that of the cross-sectional area of the portion adjacent to the proximal end (4) of the first tube.

Inside the first tube are, for purpose of example, two tools comprising an about 3 mm diameter second tubes (5) that are attached to instruments (6), for example, scissors and forceps (7) in FIGS. 7A and 7B. For instruments such as scissors that need to open and close blades, each second tube contains an about 1.5 mm diameter push-pull rod (9). In the alternative, the tool can also be manufactured separately and attached to an optional second tube as required during use. The second and first tubes and the push-pull rods can be manufactured from a variety of compositions and compounds including, but not limited to, metals, such as stainless steel, copper, aluminum, or the like, polymeric compounds, such as such as DELRIN (acetal), acrylonitrile butadiene styrene (ABS), nylon, polypropylene, polybromate, polycarbonate, glycolised polyethylene terephthalate (PETg) copolyester, or the like, glass fiber-resin composites and the like, and that can be easily machined or injection molded to the required shape. The dimensions mentioned above can be adapted and changed for use as desired.

As shown in FIG. 7D, just above the handle are two circular stops (40) to either side of the pivoting member, and a cut-out (79) in bracket (70) near the anterior stop. The purpose of stops (40) is to keep the handle (33) from pivoting until it is raised by the knob (8) sliding forward, such that the cut-out moves up to a position where it can allow the handle (33) to rotate the needed amount in the clockwise direction. As shown in FIG. 9, at the distal end of slot (36) along which the projecting circular pin (35) travels to engage the pivoting bracket (70) hook (82), the slot turns downwards over a short distance (78). The downward slanting portion of the slot (78) enables the pin (35) to engage with the hook because as the pin is pushed forward, it induces the pin to pivot around the longitudinal axis of push-pull rod in a downward (counter clockwise) direction. This happens simultaneously with the pivoting bracket (70) being raised to engage the pin.

The cross-section of the second tube and/or the rod can be round, it can be square, it can be oblong, it can be triangular, it can be a pentagon, it can be a hexagon, it can be an octagon, it can be a polygon, it can be similar to the sector of a circle, and/or it can be designed and manufactured such that multiple tools may be positioned and stored within the lumen of the first tube in a more space-saving manner.

### Deployment and Retraction

Second tube (5) with its tool (for example, bipolar electrodes) can be deployed to its working position at the distal end of the instrument by pushing knob (8) (FIGS. 7E and 7F and FIG. 9) forward along a longitudinal slot (25) in the tube housing (10) attached to the instrument grip (11). The knob (8) is attached to second tube (5) through the link (12) shown in FIG. 7F and FIG. 10. The link (12) is held in place on second tube (5) in a way that allows the tube to be rotated freely about its longitudinal axis when desired. The link (12) also presses against a low-force compression spring (13). FIG. 7D shows a spring (13) fully compressed when second tube (5), which is attached to, for example, the bipolar electrodes, is deployed from the distal end of the instrument. The link (12) locks the tube in place when it is displaced by knob (8) into a smaller side slot (77; see FIG. 7C) perpendicular to the longitudinal slot (20) located on the top of the upper casing (21).

In order for second tube (5) and its tool (6) to move into the deployed position shown in FIG. 7A, the second tube (5) must bend or flex. The tapered distal section of the first tube (2) guides the bending of second tube (5) in a gradual manner. In the instant instrument of the invention, the tapered section can be made from a low friction plastic capable of being autoclaved repeatedly. Also, plastic is electrically insulating, which is an advantage when an electrocautery tool is deployed. If plastic is not stiff enough, then glass fiber filled plastic or metal with an electrically insulated layer can be used instead.

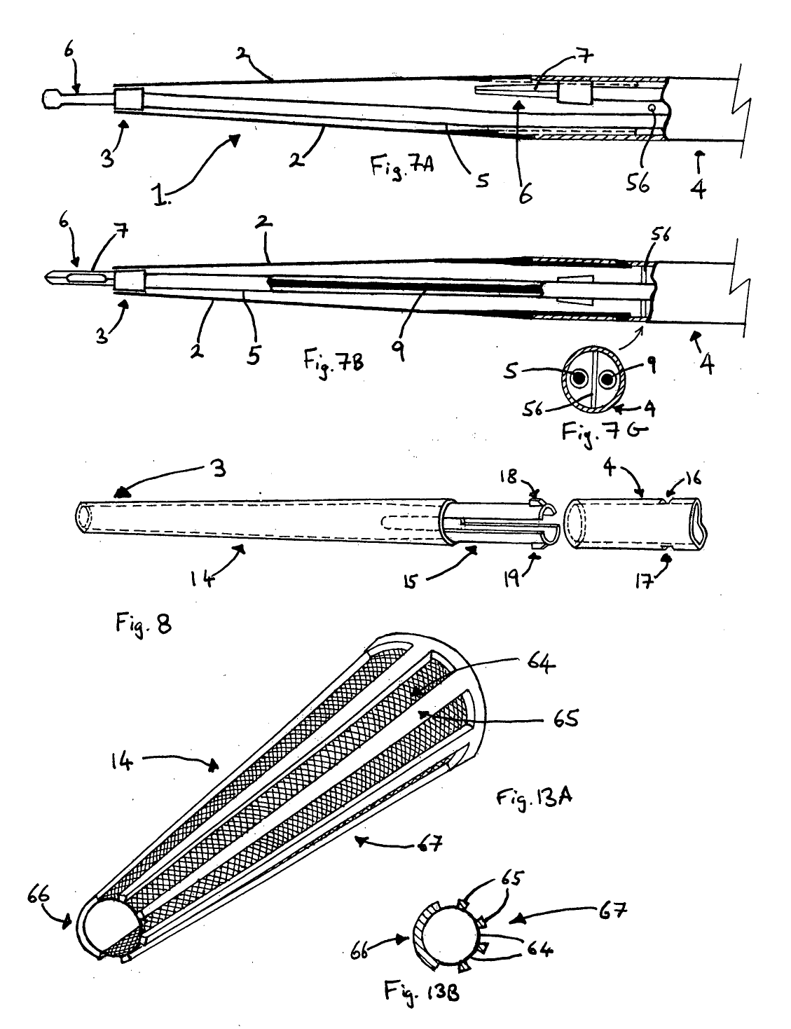

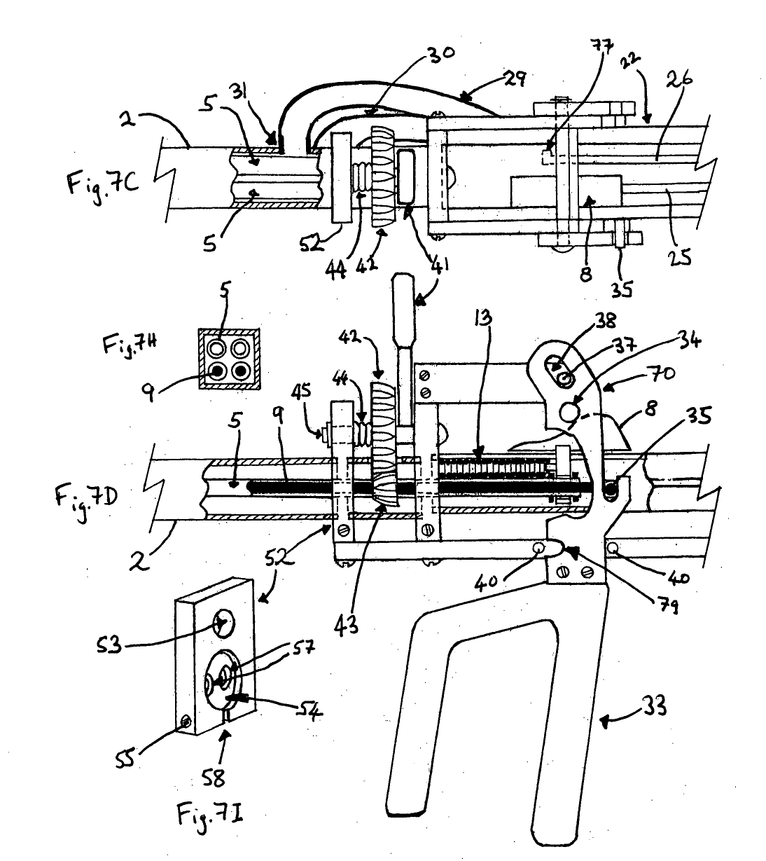

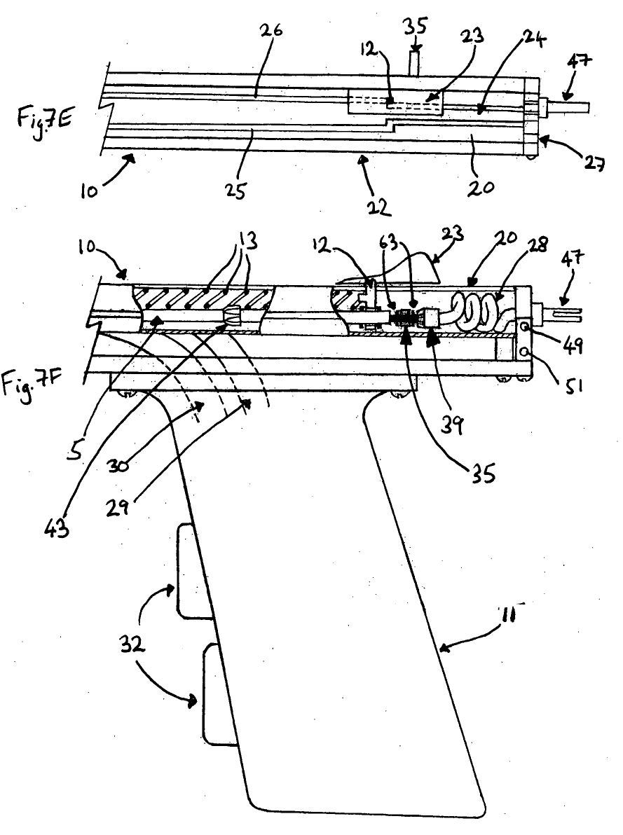

In an alternative embodiment, the tapered distal portion of the first tube (2) can comprise a separate item of manufacture that can be attached and/or removed from a non-tapered first tube, for example, as a modular tapered tube. There are numerous approaches, ways, and means that can be used to attach the tapered tubing to the first tube. For example, referring to FIG. 8, the tapered tube (14) can be made of injection molded plastic with a straight segment of tubing (15) on its end. The distal portion of first tube (3) can have two slots (16 and 17) serving two purposes. They can allow the opposing side cantilevered portions of the straight section (15) to flex inwards as the segment is slid into the proximal portion of the first tube (4). The small protrusions (18 and 19) on the opposing side portions can deflect inwards as well. When the protrusions (18 and 19) encounter the two small slots (16 and 17) on the two opposing sides of the first tube, they can deflect outwards and create what is generally known as a “snap fit.” The other purpose of the slots is to provide clearance inside the first tube for tools attached to second tubes as they are retracted or deployed. Many other means for attaching the tapered tube to a first tube may be used by one of skill in the art, such as, but not limited to, threaded screw means, adhesive means, and the like.

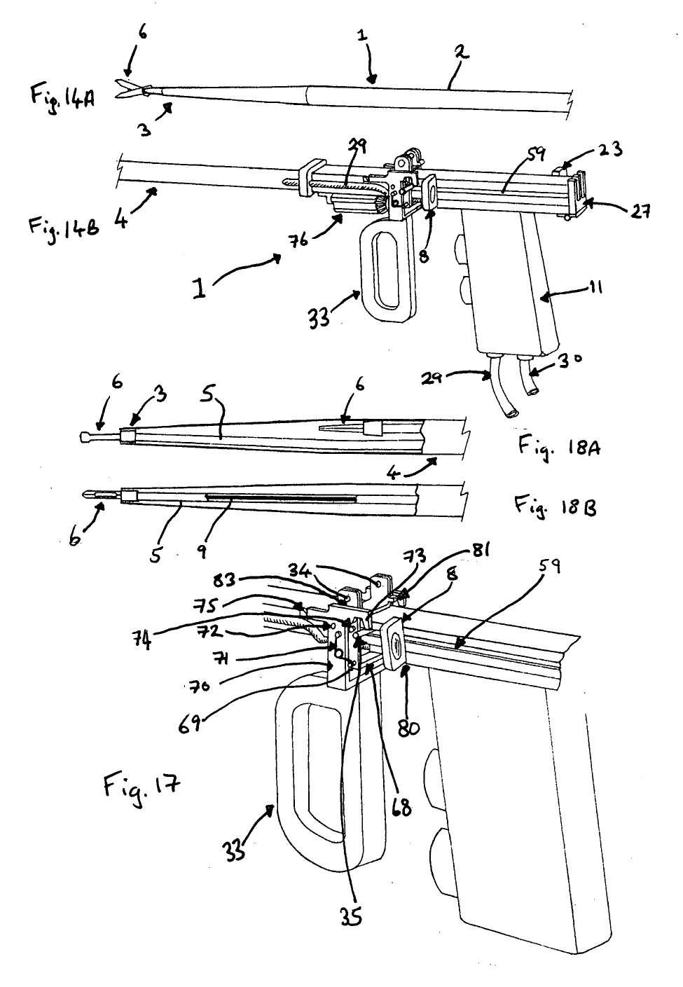

As disclosed in FIG. 13A, the tapered tube (14) can comprise a combination of a section comprising a material that is stiff and/or rigid that can deform a tool as it traverses therethrough and a section that comprises a combination of materials that are highly extensible such as elastomeric compounds (64) and a material that comprises a series of ribs (65) disposed longitudinally along the length of the tapered tube. The shape of the tapered tube and the materials used in its manufacture allow an operator to use a combination of differently dimensioned tools. In one example a first tool is advanced proximal and adjacent to the more rigid wall of the tapered tube (66) and a larger, second tool is advanced proximal and adjacent to the wall comprising the ribs and elastomeric material (67). The elasticity of the material (64) of the wall (67) allows the distal portion of the tool to be advanced and retracted within a surgical area with a reduced risk of puncture or penetration of, or damage to, the surrounding tissue than if the distal portion of the instrument were to comprise a larger diameter. In a preferred embodiment, the second tool can have about twice the cross-sectional dimensions of those of the first tool. In the alternative, the second tool can have about one-and-a-half times the cross-sectional dimensions of those of the first tool. In another alternative, the second tool can have about two-and-a-half times the cross-sectional dimensions of those of the first tool. In a still further alternative, the second tool can have about three times the cross-sectional dimensions of those of the first tool.

In the proposed design, testing of a prototype showed that the forces required to cause a second tube to flex as desired while being advanced were small. The low force levels to produce bending can be observed by manually flexing the tube of many conventional 5 mm diameter instruments to cause a deflection approximately equal to the tube diameter. In the instant instrument of the invention, the second tube is about 3 mm in diameter, which greatly reduces the forces needed to cause bending relative to that experienced in flexing a conventional instrument with a 5 mm diameter tube.

Retraction of second tube (5) can be achieved by displacing knob (8) back into the longitudinal slot (25) and letting the compressed spring move the tube back into its “storage” position. A tool (scissors in this example; 7 in FIG. 7A) attached to an additional second tube (5) is shown in the retracted position in FIGS. 7C, 7E, and 7F. It can be deployed in a manner similar to second tube (5), except by advancing knob (23) forward against the relaxed spring via its own link similar to the one shown at 12 in FIGS. 7C and 7D (deployed) and 7E and 7F (undeployed).

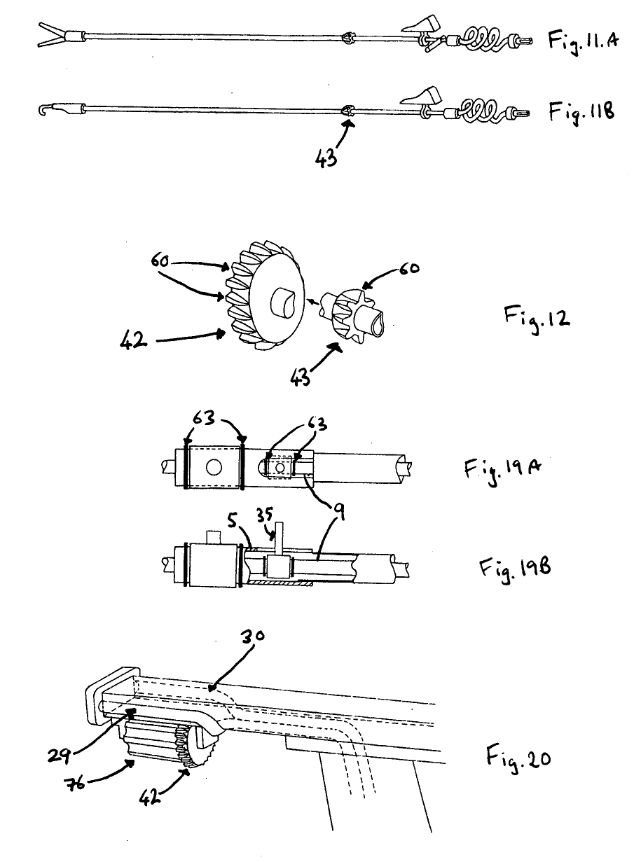

The length of the slots (25 and 26) in the top view of FIG. 7E is designed so that when a tool is retracted, there is sufficient volume and space for its push-pull rod (if it has a rod) to be in a position that corresponds to the blades of a pair of scissors being closed, as may be the case when a tool such as a scissors is retracted. The right hand ends of the slots (20 and 24) are also designed so that if the end plate (27) of the instrument is pivoted out of its locked position, it may be possible to quickly pull out an entire module (comprising a second tube (5) and the tool (6), link mechanism (12), knob (23), electrical wire (28), and terminal (47) attached to it) as illustrated in FIGS. 7E, 7F, and 9. Modules can have scissors, J-hook electrocautery, as illustrated in FIGS. 11A and 11B, or other tools known to those of skill in the art. By moving a knob from the retracted tool position into an offset slot (20 and 24), the link attached to a second tube can disengage from its spring and a module can be removed and replaced with another type of module. It is more convenient to swap tools by changing modules rather than trying to disconnect and connect tools from a second tube and push-pull rod at the distal end of the instrument.

Unless operated by an operator, the multifunctional instrument can be operated by means of an electronic control, not illustrated, which can be disposed outside the operator hand grip (33) and can be connected thereto by a cable. The control commands are received by the electronic control either via a key pad which is disposed on the casing (10) or the operator hand grip (33) or via a voice-input control. In particular, the electronic control may be as disclosed in United States Patent Application No. 2004/0249366 A1 In the case of all of the embodiments, sensors, not illustrated in detail, detect the positions of the respective instruments. This positional data is processed in the electronic control and, in accordance with the control commands input via the key pad or voice-input facility, the corresponding electromotors are then actuated. In the case of a voice-control facility, this is performed on an external computer which is then able to communicate the corresponding commands to the electronic control located in the casing (10) or communicate them directly to the electromotors.

Deployment and/or retraction of tools within the instrument may be actuated by pneumatic means, for example, bellows, electrical means, for example, motors or actuators, and/or hydraulic means, for example, pistons, and by other force transmitting mechanisms as are known in the art. Similarly, the rotation of the tools within the instrument may be actuated by such means and the like.

Modular units and other attachable/retachable/detachable elements of the instrument may require water-resistant and/or fluid-resistant seals at the area of attachment. Such seals can include, for example, labyrinth seals and the like.

### Suction/Irrigation

To allow for suction or irrigation to be applied, the tools (6) may be retracted to allow the distal end of the first tube (2) to be unobstructed. That should create sufficient space between the first tube (2) and the second tubes (5) and retracted tools (6), as shown in insets in FIGS. 7B and 7C, to allow fluids to be transported to and from the external tubes (29, 30) attached to the first tube at the location (31) shown just in front of the grip region. As illustrated in FIGS. 7F and 9, the external tubes may be routed into the grip (11) so that suction or irrigation could be activated from push buttons (32) attached to valves within the grip (11). Lines from the valves may exit through the bottom of the grip.

### Actuation of Tools

To cause the blades of a scissors, forceps, etc. to open or close, the handle (33) as shown in FIG. 7D and FIG. 9 may be used. To close the blades, one or two fingers may pull on the handle to rotate it in a counterclockwise direction around fixed pin (37) shown in FIG. 7D and FIG. 9. To open the blades, finger pressure may be exerted in the opposite direction to pivot the handle in a clockwise direction.

Pivoting the handle may cause the blades to open and close in the following way. Suppose that the tool attached to second tube (5) is to be deployed. Knob (8) shown above the grip may be pushed forward. As it does so, a small pin (see FIGS. 7C, 7E and FIG. 9; 35) attached to the end of the push-pull rod (9) for second tube (5) also may move forward through the slot (36) shown in the side housing (22). The push-pull rod (9) is not rigidly attached to the small pin (35), but could rotate freely as a tool, second tube, and push-pull rod were rotated about the longitudinal axis to different angular orientations.

As knob (8) is moved toward its fully deployed position, its wedge shape may cause pin (34) attached to the handle (33) mechanism to move upwards toward fixed pin (37) via a slot (38) shown in FIG. 7D and FIG. 9. This action, would, in turn, cause the handle (33) to move upwards and to pivot in a clockwise direction, engaging the small rod as depicted in FIG. 7D for the fully deployed position. Engagement is assisted by the pin (35) rotating about the longitudinal axis of the push-pull rod (9) as it enters the downward pointing termination of the slot (78) as shown in FIG. 9. Pivoting the handle (33) may then cause the push-pull rod to move back and forth to close or open blades in a controllable manner, by way of a retaining rings (63) on either side of the small rod and the push-pull rod (9) as shown in FIG. 10A (for knob (8) in the illustration). When released from its fully deployed position, knob (8) may be pushed backwards by the force of the compressed spring (13). The handle may drop down and be free to pivot in a counterclockwise direction. The blades may then close automatically when being retracted. Similar opening and closing of a tool attached to second tube (5) could be obtained using knob (23). Small stops (40) shown in FIG. 7D and FIG. 9 limit the rotational pivoting of the handle (33) until it is raised into a position by the action of the knob (8) so that the cut-out (79) shown in FIGS. 7D and 9 allows pivoting in a clockwise direction (when viewed from the left side).

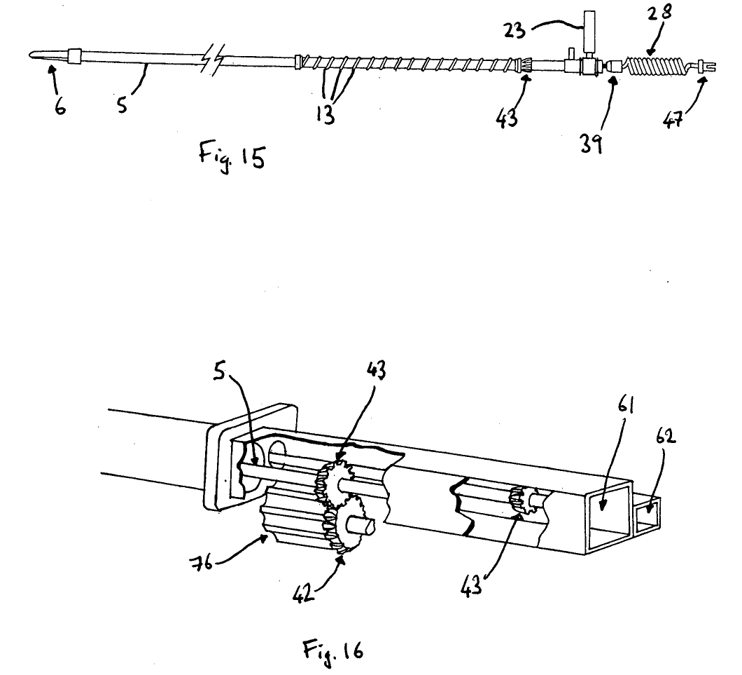

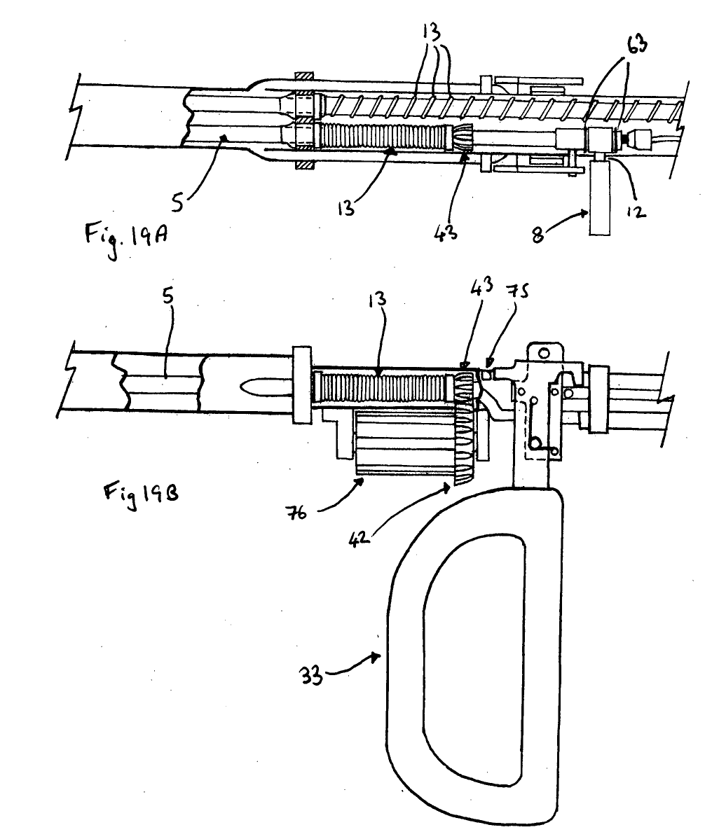

An alternative embodiment of the invention is disclosed in FIGS. 13-20. In this embodiment the instrument can be operated using a single hand, the various knobs, levers, etc. being positioned more proximal to the operator. In this alternative embodiment, as depicted in FIGS. 14B and 17, the instrument (1) comprises a first tube (2) of about 10 mm diameter wherein the distal portion of the tube tapers over a length of about 8 cm to a diameter of about 5 mm at the distal end (3) of the instrument. The distal end of the first tube has a cross-sectional area selected from the group consisting of about one half, about one third, about one quarter, about one fifth, about one sixth, about one eighth, about one tenth, and any intermediate values thereof, to that of the cross-sectional area of the proximal end (4) of the first tube.

Illustrated disposed within the lumen of the first tube (2) is, for purpose of example, a tool comprising an about 3 mm diameter second tube that is attached to a surgical instrument (6), for example, scissors (7) in FIG. 14A. In the alternative, the tool can also be manufactured separately and attached to an optional second tube as required during use. The second and first tubes and the push-pull rods can be manufactured from a variety of compositions and compounds including, but not limited to, metals, such as stainless steel, copper, aluminum, or the like, polymeric compounds, such as such as DELRIN (acetal), acrylonitrile butadiene styrene (ABS), nylon, polypropylene, polybromate, polycarbonate, glycolised polyethylene terephthalate (PETg) copolyester, of the like, glass fiber-resin composites and the like, and that can be easily machined or injection molded to the required shape. The dimensions mentioned above can be adapted and changed for use as desired.

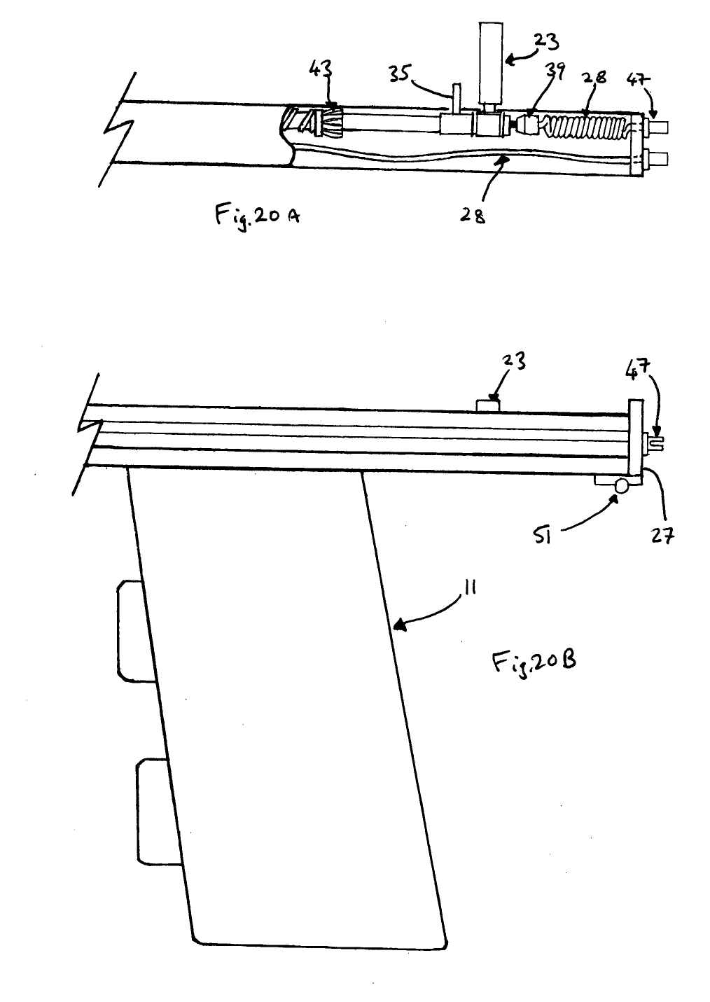

The disposition of the control elements in this alternative embodiment allows an operator to use and operate the instrument with one hand. FIGS. 14B and 15 illustrate examples of this alternative embodiment. The proximal portion of the instrument or device comprises a second tube (5) with its tool (6; for example, bipolar electrodes) can be deployed to its working position at the distal end of the instrument by pushing knob (8) (FIG. 14B) forward along a slot (59) in the tube housing (10) attached to the instrument grip (11). The second tube also comprises a low-force compression spring (13). FIGS. 19A and 19B shows a spring (13) fully compressed when second tube (5) is deployed from the distal end of the instrument. FIG. 19A also illustrates a second tube in an undeployed position showing an uncompressed compression spring.

FIG. 17 illustrates a mechanism that may be used to control the movement of the tool within the instrument. As the knob (8) is advanced towards the distal portion of the instrument, pin (35) advances through slot (59) until it reaches bracket (68) at which point travel of the knob (8) is halted and it is secured into position by the operator rotating knob (8) into a notch (80). Bracket (68) is held in place to the body of the instrument by pins (34) that allows the bracket to rotate about pins (34). In addition, an inner U-shaped pivoting member (83) comprising two vertical pieces attached at the bottom with a horizontal piece is positioned within the parallel vertical members of bracket (68). To keep that member (83) from pivoting in a counterclockwise direction beyond a certain point, there is a rectangular stop (81) in the support structure on top of the instrument about which the U-shaped member (83) can pivot. When the handle (33) is rotated in a clockwise direction by an operator or by a motor or the like, it causes bracket (70) to pivot about pin (72) and to become free of stop (75). As bracket (70) rotates, it allows ratchet (73) to clip over pin (35). Further rotation of bracket (70) relative to bracket (68) is prevented by pin (74) on bracket (68). Continued rotation of handle (33) in a clockwise direction causes bracket (68) to rotate in that direction and pulls pin (35) towards the distal end of the instrument. This, in turn, causes the push-pull rod (9) to displace (advance) towards the distal end of the tool, thereby operating the opening of scissor blades, forceps, clip, or the like.

The handle (33) can be rotated in a counter-clockwise direction to then cause the push-pull rod (9) to displace in the opposite direction (retract) and thus close the scissor blades, forceps, clips, etc. This causes bracket (68) to rotate about pin (34) and to press pin (35) towards the proximal end of the instrument. Rotation of the handle (33) in the counter-clockwise direction also causes ratchet (73) to disengage from pin (35). Rotation of the handle (33) in the clockwise direction will re-engage the ratchet (73). To release and undeploy the tool, the operator or motor or the like rotates the knob (8) out of and away from notch (80) and allows the action of a compressed spring (or other compression and expansion means) (13) to move the tool back into its resting position for storage in the instrument.

When the handle (33) is not being used, stops (75) and (80) working in concert with torsion spring (71) maintains the handle (33) in place ready to be used.

Other ways of causing a push-pull rod to actuate a tool could be used instead of that described above.

Rotation of Tools about their Longitudinal Axes

To cause a deployed tool to rotate about its longitudinal axis to a desired orientation, an arm (41) shown in FIG. 7D and FIG. 9 may be rotated in a plane perpendicular to the plane in which handle (33) rotates. The arm (41) is attached to a specially shaped larger gear (42; first gear) that engages a specially shaped smaller gear (43; second gear) attached to second tube (5). Engagement of the two gears occurs when a tool is deployed, i.e., the smaller gear on second tube (5) slides to the left to contact the teeth of the larger gear. When a tool is retracted, the gear (43) on a second tube disengages and moves to the right, as shown in FIG. 7F for second tube (5). To help the tapered gears maintain engagement, a compression spring (44) or the like shown just to the left of the larger gear mounted upon a spindle (45) presses the gear to the right.

The larger gear, compression spring, and arm assembly are positioned upon the instrument using a plate (52), the plate (52) comprising an aperture (53) to retain the larger gear and arm assembly, a recess (54), the recess (54) further comprising at least two apertures (57) through which are each passaged a second tube (5) (see FIG. 7D, inset, and 9). The plate may comprise a slit (58) that can allow an operator to mount the plate upon the instrument. A retaining screw (55) may be used to adjust the grip of the plate upon the second tubes.

The gears can comprise engagement teeth that are shaped and adapted to facilitate engagement of the two gears irrespective of their orientation to one another. As disclosed in FIG. 12, the engagement teeth (60) comprise a doubly tapered shape wherein the tip of the taper of one gear (42) can engage with the tip of the taper of a second gear (43). As the operator (or motor) continues to change the position (advance or retract as shown by arrow) of the tool within the lumen of the instrument, the gears can engage fully and operate as disclosed herein.

FIG. 16 illustrates an exemplary arrangement of the gears in the alternative embodiment of the invention, whereby the gears are closer to the proximal portion of the instrument. This figure also illustrates another alternative embodiment whereby the at least two first tubes comprise different cross-sectional dimensions (61 and 62). FIGS. 14B and 19B show a knob (76) that may be used to rotate the gear (42). Rotation of gear (42) when gear (43) is engaged, transmits movement to the second tube and tool, thereby enabling an operator to adjust the orientation of the tool in the surgical area. Rotation can also be performed remotely using pneumatic, electrical, and/or hydraulic means or the like as disclosed herein.

The diameter of the larger gear is, for example, four times that of the smaller gear so that rotating the arm to different positions through an arc of 90 degrees will cause a second tube and the tool attached to it to rotate to any desired position within a full 360 degree arc. The spring force that presses against the larger gear can be selected to provide sufficient friction to allow a desired angular orientation to be maintained once it is reached. Where the diameter of the first gear is five times the diameter of the second gear, the arm is rotated through an arc of 72 degrees to provide tools with a full 360 degree arc. Where the diameter of the first gear is three times the diameter of the second gear, the arm is rotated through an arc of 120 degrees to provide tools with a full 360 degree arc. Those of skill in the art are familiar with how many degrees of arc to rotate through in cases where the diameter of the first gear is twice that of the second gear and where the diameter of the first gear is six times that of the second gear.

### Electrical Connections

A number of ways could be used to provide electrical current to tools. One will be described here. As illustrated on FIGS. 7A, 7E, 7F, 9, 10A, 14B, 15, 20A, and 20B, second tube (5) comprises a tool (6) further comprising scissors with a monopolar electrocautery capability. Electrical current may be supplied by a wire (46) connected to a terminal (47) projecting from a slot (48) in the end plate (27), as shown in FIG. 9. On the opposite side of the end plate, the wire (28) may be coiled in a manner similar to wires used on desk telephones, as depicted in FIG. 10A. The coiled wire (28) may be connected to a device shown in FIG. 10B using interconnection means (39) that may transfer current to an insulated push-pull rod (9) in a way that may allow the push-pull rod (9) to be rotated freely about is longitudinal axis. During fabrication of a module, the connecting device may be attached to the push-pull rod by bending small tongues of conducting metal so that they may be able to contact a small circular disc attached to the end of the push-pull rod. The means can further comprise an insulating sleeve to reduce the risk of electrical short circuit to other parts of the instrument and to comply with safety standards. FIG. 20A illustrates the wire (28) in a coiled arrangement (tool undeployed) and an uncoiled arrangement (tool deployed). FIGS. 14B and 20B illustrate a hinge (75) further comprising a torsion spring (not shown) that exerts force on the end plate (27) against the proximal end of the instrument, thereby maintaining the end plate in a “closed” state. The end plate may be opened manually by the operator or by other means as disclosed herein.

Since second tubes and their push-pull rods may move back and forth when tools were deployed or retracted, the coiled configuration of the wire (28) shown in FIGS. 10A and 20A may accommodate deployment of a tool and also prevent bunching up and tangling of a wire when a tool was retracted. The spring action of the coiled wire when a tool is deployed may also pull the metal tongues (39) shown in FIG. 10B against the circular disc (81) on the push-pull rod to help maintain current flow between the two.

An electrical connection providing two wires instead of one could be used for a bipolar tool, such as shown on second tube (5), of the instrument. The connection is not shown here to reduce clutter in the drawing.

### Installing and Removing Modules

A module consisting of a second tube (5), tool (6), push-pull rod (9; if needed) and the several devices attached to the tube (see, for example, FIG. 9), including a knob (8, 23), could be removed or installed by swinging open or closing the end plate (27) about a pivot pin (51) the end plate acting as a gate. The end plate (27) may be held in place by a ball detent (49) in a frame (50) surrounding a portion of the end plate. (The detent is hidden from view in the drawing.) The slots (48) may allow the end plate to pivot open with electrical wires present, so that a module could removed or installed.

### Other Aspects of Instrument

Another aspect of the instrument is the use of guide pins (56) at several points along the first tube (see FIGS. 7A and 7B and inset). The purpose of the pins to keep the second tubes in position and to serve as anti-buckling guides. In other words, when a second tube is being moved forward under manually applied force, the pins can help prevent second tubes from deflecting too much in a direction transverse to the direction of movement. A given guide pin can be installed by drilling a hole through both sides of a first tube and using epoxy adhesive or a press fit or other means to hold the pin in place. (Several epoxy adhesives are capable of being autoclaved repeatedly.)

The instrument shows the use of retaining rings (63) in FIGS. 10A and 19A and 19B to join link (12) and the small pin (35) to a push-pull rod (9) in a manner that may allow the push-pull rod to rotate freely. Such small retaining rings are commercially available, as are the small ball detents used in the design.

### Variations and Modifications

Additional uses and methods of use are described below:
(a) It may be possible to modify the instrument to have three or more modules rather than the two shown in this description.
(b) Modules could be advanced or retracted pneumatically or by other powered means instead of manually.
(c) Modules could be advanced or retracted or rotated or interchanged using robotic devices.
(d) The instrument is not limited to the type of tools shown here. Other types of tool combinations could be used as well. For example, scissors plus J or L hook electrocautery, or forceps with bipolar electrocautery capability plus a monopolar electrocautery tool, and so forth (see FIG. 11).
(e) Rather than having a fixed first tube as in the approach described here, it may also be possible to have an instrument in which the first tube is rotated so that, when deployed to the center of the distal end of the first tube, a tool may also rotate to a desired orientation—an approach commonly used with existing single purpose instruments: An advantage of this approach is that it may avoid possible problems associated with rotating a second tube holding a tool using gears or other means. Suction or irrigation could be provided by way of the first tube, but with a stationary chamber connected to it and to external suction/irrigation lines in the vicinity of the grip. The chamber may allow fluid from the first tube to be transported into or out of it for any rotational orientation of the first tube. Opening and closing or blades, forceps, etc. may be achieved via push-pull rods inside second tubes. A mechanism may be provided to enable a handle to move the push-pull rods back and forth for the numerous different positions of the rods that may result from different rotational orientations of the first tube.
(f) The various modules and individual components of the instrument can be manufactured so as to be autoclavable or otherwise sterilizable. In the alternative, such modules and components can be manufactured so as to be disposable and/or biodegradable.

### LIST OF REFERENCE NUMERALS

1. Multifunctional Surgical Instrument
2. First Tube
3. Distal End Portion of First Tube
4. Proximal End Portion of First Tube
5. Second Tube
6. Tool or Instrument
7. Scissors or Forceps
8. Knob
9. Push-Pull Rod
10. Tube Housing or Casing
11. Grip
12. Link
13. Spring
14. Tapered Tube
15. Straight Segment of Tubing
16. Upper Slot
17. Lower Slot
18. Upper Protrusion
19. Lower Protrusion
20. Longitudinal Slot
21. Upper Casing or Housing
22. Sidewall Casing or Housing
23. Knob
24. Longitudinal Slot
25. Longitudinal Inner Slot
26. Longitudinal Inner Slot
27. End Plate
28. Electrical Wire
29. External tube for Fluid Transport (e.g. irrigation tube)
30. External Tube for Fluid Transport (e.g. vacuum tube)
31. Location for Attaching External Tube(s)
32. Push Buttons to Regulate Flow of Fluid in external Tube(s)
33. Hand Grip or Handle
34. Pin
35. Pin
36. Side Slot
37. Pin
38. Slot
39. Connecting Means (e.g. metal tongues, insulating nut)
40. Stop
41. Arm
42. First Gear
43. Second Gear
44. Compression Spring
45. Spindle
46. Wire
47. Electrical Terminal
48. Slot
49. Ball Detent
50. Frame
51. Pivot Pin
52. Plate
53. Aperture
54. Recess
55. Retaining Screw
56. Guide Pin
57. Aperture
58. Slit
59. Side Slot
60. Teeth
61. Larger Cross-Section of Tube
62. Smaller Cross-Section of Tube
63. Retaining Ring
64. Portion of Tubing Comprising Elastomeric Material
65. Rib of Tubing
66. Rigid Wall of Tapered Tube
67. Wall Having Ribs and Elastomer Material
68. Bracket
69. Pin
70. Bracket
71. Torsion Spring
72. Pin
73. Ratchet
74. Stop Pin
75. Stop Pin
76. Knob
77. Side Slot
78. Curved Portion of Slot End
79. Cut Out in Bracket
80. Notch
81. Circular Disc
101. Trocar
102. Endoscope
103. Surgical Field
104. Laparoscopic Surgical Instrument
201. Laparogcopic Instrument
202. Scissors
203. Hollow Tube
204. Push-Pull Rod
205. Grip
206. Linkage
207. Knob
300. Rod
301. Electrode
302. Channel for Suction or Irrigation
303. Channel
304. Telescope
305. Wire
306. Electrode
307. Irrigation and Suction Tube
308. Shunt Tubing
309. Outer Tube
310. Lumen of Outer Tube

Although the various exemplary embodiments of the present invention are directed to medical laparoscopic uses, the present invention is not limited to such uses, and the instrument described may be used for any application in which it is important to maintain an instrument in a particular location or site. Such instruments may be used to perform tasks or procedures in machinery or devices that are relatively inaccessible, thereby reducing time and costs spent disassembling the machinery or device. The instrument of the invention solves such problems in the same way as described above for the laparoscopy examples.

Those skilled in the art will appreciate that various adaptations and modifications of the just-described embodiments can be configured without departing from the scope and spirit of the invention and the above description is intended to be illustrative, and not restrictive, and it is understood that the applicant claims the full scope of any claims and all equivalents.

1. A multifunctional surgical instrument, the instrument comprising a body and a plurality of tools, the body defining a first lumen and the tools disposed therewithin and wherein the body further comprises a tapered distal portion.

2. The multifunctional surgical instrument of claim 1, wherein the tapered distal portion comprises at least one percent of the length of the body and wherein the tapered distal portion has a distal end and a proximal end.

3. The multifunctional surgical instrument of claim 2, wherein the distal end of the tapered distal portion has a cross-sectional area smaller than that of the cross-sectional area of the proximal end of the tapered distal portion.

4. (canceled)

5. (canceled)

6. (canceled)

7. (canceled)

8. The multifunctional surgical instrument of claim 1, wherein the tool has a distal portion and a proximal portion and comprises a flexible material.

9. (canceled)

10. (canceled)

11. (canceled)

12. (canceled)

13. (canceled)

14. (canceled)

15. The multifunctional surgical instrument of claim 8, wherein the tool flexes at the distal portion of the tool.

16. (canceled)

17. (canceled)

18. The multifunctional surgical instrument of claim 1, wherein the tool is selected from the group consisting of scissors, forceps, spoon forceps, graspers, a clip, clip applier, clip applicator, dissector, stapler, hook, laser-emitting device or laser delivery system, an obturator, cauterizing wire, electrocautery devices including monopolar and bipolar instruments, glue gun, suction tubing, pressure tubing, and irrigation tubing.

19. (canceled)

20. (canceled)

21. The multifunctional surgical instrument of claim 1, wherein the tool is shaped and adapted for advancing, retracting, and rotating within the lumen of the body.

22. The multifunctional surgical instrument of claim 1 wherein the tool is adapted for use with an electric operating system.

23. The multifunctional surgical instrument of claim 1 wherein the tool is adapted for passage of fluids.

24. The multifunctional surgical instrument of claim 23 wherein the adaptation for passage of fluids comprises tubing selected from the group consisting of irrigation tubing and suction tubing.

25. The multifunctional surgical instrument of claim 1 wherein the tools are selected from the group consisting of disposable tools and re-usable tools.

26. (canceled)

27. (canceled)

28. The multifunctional surgical instrument of claim 1 wherein the instrument is adapted for use with one hand.

29. (canceled)

30. The multifunctional surgical instrument of claim 1 comprising tools having different dimensions.

31. (canceled)

32. The multifunctional surgical instrument of claim 1 wherein the tapered distal portion comprises different materials, the different materials having different physical properties.

33. (canceled)

34. (canceled)

35. (canceled)

36. (canceled)

37. The multifunctional surgical instrument of claim 1, further comprising a pair of tapered gears, wherein the gears are shaped and adapted to engage with one another whilst the tool is rotating.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. A multifunctional surgical instrument having at least one tool, the instrument having a single distal aperture and a single proximal aperture, wherein the instrument is tapered between the single distal aperture and the single proximal aperture thereby providing a tapered distal end portion and wherein the tool, in use, reversibly protrudes from the single distal end.

51. (canceled)

52. The multifunctional surgical instrument of claim 50 further comprising means adapted for passage of fluids.

53. The multifunctional surgical instrument of claim 52 wherein the means adapted for passage of fluids comprises tubing selected from the group consisting of irrigation tubing and suction tubing.

54. (canceled)

55. (canceled)

56. (canceled)

57. The multifunctional surgical instrument of claim 50, further comprising a pair of tapered gears, wherein the gears are shaped and adapted to engage with one another whilst the tool is rotating.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. The multifunctional surgical instrument of claim 50, wherein the tool is selected from the group consisting of scissors, forceps, spoon forceps, graspers, a clip, clip applier, clip applicator, dissector, stapler, hook, laser-emitting device or laser delivery system, an obturator, cauterizing wire, electrocautery devices including monopolar and bipolar instruments, glue gun, suction tubing, pressure tubing, and irrigation tubing.
